(19) United States
(12) Patent Application Publication
(10) Pub. No.: US 2010/0233575 A1
(43) Pub. Date: Sep. 16, 2010

(54) QUATERNARY AMMONIUM COMPOUNDS AND THEIR USES

(75) Inventors: Ian David Brotherston, Malvern (GB); John Lindley Bancroft, Cheshire (GB)

Correspondence Address: KING & SCHICKLI, PLLC, 247 NORTH BROADWAY, LEXINGTON, KY, 40507, US

(73) Assignee: IONIC POLYMER SOLUTIONS LIMITED, Malvern (GB)

(21) Appl. No.: 12/294,439

(22) PCT Filed: Mar. 26, 2007
(86) PCT No.: PCT/GB2007/001084

§ 371 Date: Nov. 26, 2008

(30) Foreign Application Priority Data
Mar. 25, 2006 (GB) 0606016,4

(51) Int. Cl. C07C 211/63; H01M 8/10; C25D 5/00; C25F 7/00; H01M 8/08; C25B 13/00; H01B 1/12; C10L 1/22; B01D 15/00

(52) U.S. Cl. 429/491; 564/295; 205/80; 205/704; 205/770; 429/498; 204/296; 252/500; 508/547; 210/660

(57) ABSTRACT

According to the invention there is provided a method of conduction including the steps of providing a quaternary ammonium compound, and causing the quaternary ammonium compound to conduct ionically.

This invention relates to quaternary ammonium compounds and their uses. Particular, but by no means exclusive, reference is made to methods of ionic conduction, compounds suitable for ionic conduction, and structures incorporating such compounds. Reference is also made to the provision of ionic liquids and methods of dissolving substances.

There is much interest in the manufacture and use of ionically conductive substances. A wide range of ionically conductive polymers are known, with possibly the most famous example being Nafion®. However polymers, by their nature, tend to be solid materials. The present invention provides a class of non-polymeric, ionically conductive compounds, the physical properties of which may be tailored to the desired end application. In particular, it is possible to provide ionically conductive liquids that can be used as liquid electrolytes. Other applications are disclosed also.

According to a first aspect of the invention there is provided a method of conduction including the steps of providing a quaternary ammonium compound, and causing the quaternary ammonium compound to conduct ionically.

Preferably, the quaternary ammonium compound is an ionic liquid. Ionic liquids are generally understood to be salts which are liquid below 100° C. Some embodiments of the invention are room temperature ionic liquids, i.e. salts which are liquid at or below 25° C. The quaternary ammonium compound may be a liquid at 20° C. and atmosphere pressure. Alternatively, the quaternary ammonium compound may be a solid under these conditions.

The quaternary ammonium compound may be provided in the form of an admixture with a solvent. The solvent may assist in producing a liquid admixture, and can confer benefits over the use of the pure quaternary ammonium compound and the pure solvent. Examples of solvents are water, alcohols and propylene carbonate. Typically, the admixture contains less than 10%, and preferably less than 5% of the solvent by weight, although higher loadings of solvent, for example up to 25% or greater, are possible.

The quaternary ammonium compound may conduct by anionic and cationic conduction, which may involve proton conduction.

International Publications WO00/06610, WO00/06533, WO00/06658, WO01/36510, WO01/40874 and WO01/74919, the contents of all of which are herein incorporated by reference, disclose a class of polymers obtained from the polymerisation of a number of compounds which possess one or more dienyl end groups. These documents are principally concerned with the properties of polymers disclosed therein, although the preparation of the corresponding monomers is described. The present inventors have surprisingly found that some of the monomers corresponding to polymers disclosed generally in the aforementioned International Publications can conduct ionically and/or possess other useful properties and applications which are described herein.

Preferably, then, the quaternary ammonium compound is a dienyl quaternary ammonium compound, most preferably comprising a group of sub-formula (I)

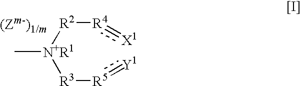

where R² and R³ are independently selected from (CR⁷R⁸)ₙ, or a group CR⁹R¹⁰, CR⁷R⁸CR⁹R¹⁰ or CR⁹R¹⁰CR⁷R⁸ where n is 0, 1 or 2, R⁷ and R⁸ are independently selected from hydrogen, halo or hydrocarbyl, and either one of R⁹ or R¹⁹ is hydrogen and the other is an electron withdrawing group, or R⁹ and R¹⁰ together form an electron withdrawing group, and

R⁴ and R⁵ are independently selected from CH or CR¹¹ where R¹¹ is an electron withdrawing group;

the dotted lines indicate the presence or absence of a bond, X¹ is a group CX² X³ where the dotted line bond to which it is attached is absent and a group CX² where the dotted line bond to which it is attached is present, Y¹ is a group CY²Y³ where the dotted line bond to which it is attached is absent and a group CY² where the dotted line bond to which it is attached is present, and X², X³, Y² and Y³ are independently selected from hydrogen and fluorine;

and R¹ is hydrogen or hydrocarbyl, and Z is an anion of charge m.

The properties of the compound can be varied and tailored to suit the desired application by varying the nature of the anion and the substituents pendant from the quaternary nitrogen. This enables the provision of “task specific” ionic liquids.

Preferably, R⁷ and R⁸ are independently selected from fluoro, chloro, alkyl or H. In the case of alkyl, methyl is most preferred. Compounds in which both R⁷ and R⁸ are methyl have been prepared, and have been found to be stable at high temperatures.

In preferred embodiments, X¹ and Y¹ are groups CX² X³ and CY¹Y² respectively and the dotted lines represent an absence of a bond. Thus preferred compounds are those of sub-formula (IA)

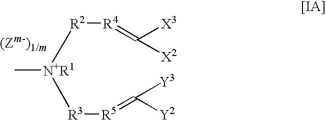

where R¹, R², R³, R⁴, R⁵, R⁶, X², X³, Y² and Y³ are as defined above.

As used herein, the term “alkyl” refers to straight or branched chain alkyl groups, suitably containing up to 20 and preferably up to 6 carbon atoms. The term “alkenyl” and “alkynyl” refer to unsaturated straight or branched chains which include for example from 2-20 carbon atoms, for example from 2 to 6 carbon atoms. Chains may include one or more double to triple bonds respectively. In addition, the term “aryl” refers to aromatic groups such as phenyl or naphthyl.

The term “hydrocarbyl” refers to any structure comprising carbon and hydrogen atoms. For example, these may be alkyl, alkenyl, alkynyl, aryl such as phenyl or napthyl, arylalkyl, cycloalkyl, cycloalkenyl or cycloalkynyl. Suitably they will contain up to 20 and preferably up to 10 carbon atoms. The term “heterocylyl” includes aromatic or non-aromatic rings, for example containing from 4 to 20, suitably from 5 to 10 ring atoms, at least one of which is a heteroatom such as oxygen, sulphur or nitrogen. Examples of such groups include furyl, thienyl, pyrrolyl, pyrrolidinyl, imidazolyl, triazolyl, thiazolyl, tetrazolyl, oxazolyl, isoxazolyl, pyrazolyl, pyridyl, pyrimidinyl, pyrazinyl, pyridazinyl, triazinyl, quinolinyl, isoquinolinyl, quinoxalinyl, benzthiazolyl, benzoxazolyl, benzothienyl or benzofuryl.

The term “functional group” refers to reactive groups such as halo, cyano, nitro, oxo, C(O)ₙR^{a}, OR^{a}, S(O)ₜR^{a}, NR^{b}R^{c}, OC(O)NR^{b}R^{b}, C(O)NR^{b}R^{b}, OC(O) NR^{b}R^{b}, —NR⁷C(O)ₙR⁶, —NR^{a}CONR^{b}R^{c}, —C═NOR^{a}, —N═CR^{b}R^{c}, S(O)ₜNR^{b}R^{c}, C(S)ₙR^{a}, C(S)OR^{a}, C(S)NR^{b}R^{c} or —NR^{b}S(O)ₜR^{a} where R^{a}, R^{b} and R^{c} are independently selected from hydrogen or optionally substituted hydrocarbyl, or R^{b} and R^{c} together form an optionally substituted ring which optionally contains further heteroatoms such as S(O)ₛ, oxygen and nitrogen, n is an integer of 1 or 2, t is 0 or an integer of 1-3. In particular the functional groups are groups such as halo, cyano, nitro, oxo, C(O)ₙR^{a}, OR^{a}, S(O)ₜR^{a}, NR^{b}R^{c}, OC(O)NR^{b}R^{c}, C(O)NR^{b}R^{c}, OC(O)NR^{b}R^{c}, —NR⁷C(O)ₙR⁶, —NR^{a}CONR^{b}R^{c}, —NR^{a}CSNR^{b}R^{c}, C═NOR^{a}, —N═CR^{b}R^{c}, S(O)ₜNR^{b}R^{c}, or —NR^{b}S(O)ₜR^{a} where R^{a}, R^{b} and R^{c}, n and t are as defined above.

The term “heteroatom” as used herein refers to non-carbon atoms such as oxygen, nitrogen or sulphur atoms. Where the nitrogen atoms are present, they will generally be present as part of an amino residue so that they will be substituted for example by hydrogen or alkyl.

The term “amide” is generally understood to refer to a group of formula C(O)NR^{a}R^{b} where R^{a} and R^{b} are hydrogen or an optionally substituted hydrocarbyl group. Similarly, the term “sulphonamide” will refer to a group of formula S(O)₂NR^{a}R^{b}.

The nature of any electron withdrawing group or groups additional to the amine moiety used in any particular case will depend upon its position in relation to the double bond it is desired to activate, as well as the nature of any other functional groups within the compound. The term “electron withdrawing group” includes within its scope atomic substituents such as halo, e.g. fluoro, chloro and bromo.

Where R¹¹ is an electron withdrawing group, it is suitably acyl such as acetyl, nitrile or nitro.

Preferably X¹, X², Y¹ and Y_{z} are all hydrogen.

Suitable groups R^{a} include hydrogen or methyl, in particular hydrogen.

Z^{m-} may be a halide ion, a boride ion, triflate, PF₆⁻, HSO₄⁻, SO₄²⁻, H₂PO₄⁻, imide, or a carboxylic acid ester, preferably a carboxylic acid ester having an alkyl or a per-fluorinated alkyl group of greater than five carbon atoms, most preferably octanoate or per-fluoro octanoate. Also possible are other anions having hydrocarbyl or substituted hydrocarbyl moieties, including anions having branched hydrocarbyl moieties. Many other anions might be utilised.

In the group of sub-formula (I), X¹ and Y¹ may represent CX²X³ and CY²Y³ respectively, the dotted bonds being absent and X², X³, Y² and Y³ being all hydrogen.

The quaternary ammonium compound may be a compound of structure (II)

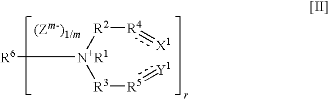

where X¹, Y¹, R², R³, R⁴, R⁵ and the dotted bonds are as defined in relation to formula (I) above, r is an integer of 1 or more, and R⁶ is a bridging group, an optionally substituted hydrocarbyl group, a perhaloalkyl group, a siloxane group or an amide, of valency r

Compounds in which r is 1 are preferred. Compounds of this type may be represented as structure (III)

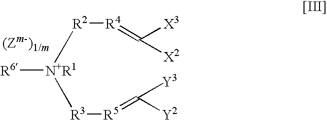

where X², X³, Y², Y³, R¹, R², R³, R⁴, and R⁵ are as defined in relation to formula (I) above, R^{6′} is an optionally substituted hydrocarbyl group, a perhaloalkyl group, a siloxane group or an amide.

Variation of the R⁶ or R^{6′} group enables the properties of the quaternary ammonium compound to be tailored to the desired application. “Task specific” ionic liquids can be produced in this way.

The invention may also be applied to other sorts of quaternary ammonium compounds; for example, where in the compounds of formula (II), r is greater than one. Particular examples are compounds of formula (II) as defined above, where R⁶ is a bridging group and r is an integer of 2 or more, for example from 2 to 8 and preferably from 2-4. Embodiments in which r is two are particularly preferred.

Examples of suitable bridging groups include those found in polymer technology, such as polyethylenes, polypropylenes, nylons, as listed in Table 1. Further examples of bridging groups can be found in WO 00/06610.

In preferred structures, R⁶ or R^{6′} comprises a straight or branched chain alkyl group, optionally substituted or interposed with functional groups.

R⁶ or R^{6′} may be an optionally substituted hydrocarbyl group having four or more carbon atoms. Preferably, R⁶ or R^{6′} is an alkyl group, most preferably a straight chain alkyl group, although R⁶ or R^{6′} may be a branched chain alkyl group. Compounds of this type can act as effective detergents, having affinity for both polar and non-polar phases. R⁶ or R^{6′} may have between five and twenty carbon atoms, preferably between eight and fourteen carbon atoms, most preferably ten carbon atoms.

In particularly preferred embodiments, the starting material is a compound of formula (IV)

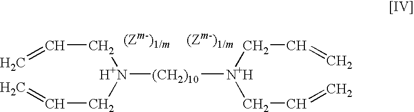

The starting material may be a compound of formula (V)

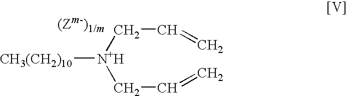

In the embodiments of formulae (IV) and (V), Z^{m-} may be PF₆⁻, per-fluoro octanoate or triflate, although the invention is not limited in this regard.

R¹ may be an alkyl group, preferably having less than three carbon atoms, most preferably methyl. Alternatively, R¹ may be H. Embodiments in which R¹ is H may be useful for providing proton conduction mechanisms.

R⁶ or R^{6′} may comprise a perhaloalkyl group, for example of from 1 to 3 carbon atoms such as a perhalomethyl group, in particular perfluoromethyl.

TABLE 1

| Polymer Type | Repeat Unit of Bridging Group |
|---|---|
| Polyethylene | CH₂ |
| Polystyrene | CH₂CH(C₆H₅) where the phenyl ring is |
| | optionally substituted |
| Polyisobutylene | CH₂CH(CH(CH₃)₂) |
| Polyisoprene | CH₂CH(CH₃) |
| Polytetrafluoroethylene | CH₂(CF₂)ₓCH₂ |
| Polyvinylidenefluoride | CH₂(CF₂CH₂)ₓ |
| Polyethyleneoxide | (OCH₂CH(CH₃))ₓ0 |
| Nylon | CH₂(NHCOCH₂)ₓCH₂ |
| Peptide | CH₂(NHCOCH_{R})ₓCH₂ |
| Polyurethanes | —NH—CO—O— |
| Polyesters | —RC(O)OR′— |
| | where R and R′ are organic groups such |
| | as hydrocarbyl |
| Polysiloxanes | e.g. —SiO₂—, —R₂SiO—or |
| | —R₂Si₂O₃—where R is an organic group |
| | such as hydrocarbyl |
| Polyacrylates | —CH₂C(COOH)H— |
| Polyureas | —NHCONH— |
| Polythioureas | —NH—C(S)—NH— |

The quaternary ammonium compound may include a substantially hydrophobic portion. Such a portion can impart detergency properties; and allow non-polar solvents to be dissolved. The substantially hydrophobic portion may be an optionally substituted hydrocarbyl group which may be an alkyl group, preferably having four or more carbon atoms, more preferably having between five and twenty carbon atoms, most preferably having between eight and fourteen carbon atoms. The substantially hydrophobic portion may be R¹, R⁶, or R^{6′}.

The quaternary ammonium compound may be located in the pores of a porous substrate, which may be a ceramic, a zeolite or a polymer. The porous substrate may be microporous. Microporous polymer substrates, such as expanded PTFE, may be used. The substrate may be a membrane.

The porous structure with quaternary ammonium compound located therein may be in the form of an ionically conductive membrane. Such conductive membranes have numerous applications, such as in fuel cells. Solid quaternary ammonium compounds are particularly suitable for such applications.

A substance may be dissolved in the quaternary ammonium compound, which may be a liquid. The substance may be a non-polar liquid, which may be a fuel, such as petroleum or diesel. Quaternary ammonium compounds that have detergency properties may be used in such applications.

Additionally or alternatively, a catalytic material may be dissolved in the quaternary ammonium compound.

In preferred embodiments, the quaternary ammonium compound is caused to conduct ionically as part of a fuel cell.

In other preferred embodiments, the quaternary ammonium compound is caused to conduct ionically in an electrochemical process. In such embodiments, a liquid quaternary ammonium compound may take the place of water or a non-aqueous solvent in an electrochemical process. The liquid quaternary ammonium compound may act as an electrolyte. Ions and/or other entities can be present in the quaternary ammonium compound as desired according to the application. Areas of application include electroplating, advantageously the electroplating of metals such as aluminium and titanium, electropolishing, electrowinning and electrosynthesis. The quaternary ammonium compound may dissolve one or more metal compounds as part of an electrochemical process.

In electrochemical processes which utilise an ionically conducting quaternary ammonium compound, said quaternary ammonium compound may dissolve gases produced by the electrochemical process. A semi-permeable membrane may be provided through which the gases can pass, thereby exiting the environs of the electrochemical process. This is advantageous since it prevents or inhibits the productions of bubbles of gas, which can cause highly undesirable fluctuations in resistance.

The quaternary ammonium compound may be deposited by electro-deposition, or may be deposited electrolessly. Electroless deposition may comprise deposition in an electroless plating bath, in which the quaternary ammonium compound is deposited without requiring the application of a potential difference across a cell or the flow of electrical current.

According to a second aspect of the invention there is provided a method of dissolving a substance including the steps of:
providing a liquid quaternary ammonium compound; and
contacting the substance with the liquid quaternary ammonium compound so that the substance is dissolved in the liquid quaternary ammonium compound.

More than one substance may be dissolved in the quaternary ammonium compound.

The substance may be polar or non-polar.

In a preferred embodiment, the substance is a catalytic material, which may be a metal, for example a precious metal such as platinum or palladium. In this way, a homogeneous catalytic process can be provided.

The quaternary ammonium compound may be as defined in the first aspect of the invention, although in some embodiments it is not necessary that the quaternary ammonium compound can conduct ionically.

The substance may be a solid, and in some preferred embodiments, the solid is an electrolyte.

Alternatively or additionally, the substance may be a gas. The ability to dissolve gases is particularly advantageous in electrochemical processes. The dissolved gas may be released from the liquid quaternary ammonium compound, and the release may be controlled, for example by varying the pressure of a gaseous atmosphere above the liquid quaternary ammonium compound.

Alternatively or additionally, the substance may be a liquid. The liquid may be a non-polar liquid. In such embodiments, the quaternary compound may be as defined in the first aspect of the invention. In particular, it is possible to provide quaternary ammonium compounds that have detergency properties suitable for dissolving non-polar liquids. Typically, the quaternary ammonium compound includes a substantially hydrophobic portion. The substantially hydrophobic portion provides detergency properties enabling the non-polar liquid to be dissolved in the quaternary ammonium compound. The substantially hydrophobic portion may be an optionally substituted hydrocarbyl group. The optionally substituted hydrocarbyl group may be an alkyl group, preferably having 4 or more carbon atoms, more preferably having between 5 and 20 carbon atoms, most preferably having between 8 and 14 carbon atoms.

Preferably the quaternary ammonium compound comprises a group of sub-formula (I) as previously defined. Most preferably the starting material comprises a compound of formula (III) as previously defined in which R⁶ and R^{6′} is an optionally substituted hydrocarbyl group.

The non-polar liquid may be a fuel, preferably petroleum or diesel.

Preferably, a fuel and a catalytic material are dissolved in the quaternary ammonium compound.

The quaternary ammonium compound may act as an electrolyte in an electrochemical process. A metal salt may be dissolved in the quaternary ammonium compound Electroplating, for example metal plating, electropolishing and electrowinning may be performed.

Alternatively, the quaternary ammonium compound may be used as the solvent in an electroless deposition process, which may be an electroless plating process, for example one in which a metal is dissolved in the quaternary ammonium compound and subsequently plated onto a surface which is in contact with the quaternary ammonium compound. Copper may be deposited in this way.

Quaternary ammonium compounds having the combined properties of ionic conductivity and the ability to dissolve non-polar liquids are advantageous, and confer utility in applications such as fuel cells.

In other embodiments, an eutectic liquid is formed by dissolving the substance in the quaternary ammonium compound. The substance may be a liquid or a solid.

Other embodiments further include the step of polymerising the quaternary ammonium compound with the substance dissolved therein. Methods for polymerising the quaternary ammonium compound can be found in International Publications WO 00/06610, WO 00/06533, WO 00/06658, WO 001/36510, WO 01/40874 and WO 01/74919. In a preferred embodiment, the substance is a waste material. Advantageously, the waste material is a liquid which is separated from the solid polymer formed by the polymerisation of the quaternary ammonium compound.

In other embodiments still, the quaternary ammonium compound is used as a solvent in a chemical or biochemical reaction, and the substance or substances are reagents in said chemical or biochemical reaction. The chemical reaction may be an organic or inorganic synthesis reaction. The biochemical reaction may involve the dissolution of an enzyme in the quaternary ammonium compound. Enzymes can die in organic solvents, but it is envisaged that quaternary ammonium compounds of the invention will be able to keep enzymes alive and allow the required reaction to take place. Application areas include biotechnology.

According to a third aspect of the invention there is provided a method of coating a surface including the step of contacting a surface with a composition including a liquid quaternary ammonium compound so as to form a coating thereon. The quaternary ammonium compound may be as defined in the first aspect of the invention.

In preferred embodiments, the composition is a paint. The composition may include pigment or other additives in order to provide a desired colour. Alternatively, the quaternary ammonium compound itself may provide the desired colour if a suitable chromophore is provided as a substituent on the quaternary ammonium compound. A preferred area of application is in the provision of “non-drying paints”, which may be utilised in anti-vandal and other applications. Many of the quaternary ammonium compounds provided by the invention have low or zero vapour pressure, and thus have utility in paints (and other coatings) that are required to stay wet. The nature of the quaternary ammonium compound can be tailored to suit the substrate upon which the paint (or other coating) is intended to be used. Hydrophobic substituents may be present on the quaternary ammonium compound in order to improve weather resistance.

In some embodiments, it is not necessary for the liquid quaternary ammonium compound to be ionically conductive. However, in other embodiments the liquid quaternary ammonium compound is ionically conductive, thereby rendering the coating conductive.

According to a fourth aspect of the invention there is provided a structure including a porous substrate and an ionically conductive quaternary ammonium compound located in the pores of the porous substrate. The quaternary ammonium compound and porous substrate may be as defined in the first aspect of the invention.

According to a fifth aspect of the invention there is provided a fuel cell including an ionically conductive quaternary ammonium compound. The quaternary ammonium compound may be as defined in the first aspect of the invention. The quaternary ammonium compound may be a solid or a liquid.

Where the quaternary ammonium compound is a liquid, the quaternary ammonium compound acts as a liquid electrolyte. The quaternary ammonium compound may be used to dissolve a liquid fuel introduced to the fuel cell. The liquid fuel may be petroleum or diesel. The quaternary ammonium compound may also have a catalyst dissolved therein, thereby permitting homogeneous catalysis to take place. Examples of catalysts include platinum and palladium.

According to a sixth aspect of the invention there is provide a liquid quaternary ammonium compound having a substance dissolved therein. The liquid quaternary ammonium compound may be as defined in the first aspect of the invention.

The substance may be a fuel.

Additionally, or alternatively, the substance may be a catalytic material.

The liquid quaternary ammonium compound may be ionically conductive.

According to a seventh aspect of the invention there is provided a coating composition including a liquid quaternary ammonium compound. The liquid quaternary ammonium compound may be as defined in the first aspect of the invention.

The coating composition may be in the form of a paint.

The liquid quaternary ammonium compound may be ionically conductive.

According to an eighth aspect of the invention there is provided a method of forming an eutectic liquid including the steps of:
providing a quaternary ammonium compound; and
mixing said quaternary ammonium compound with a substance so as to produce an eutectic liquid.

The quaternary ammonium compound may be as defined in the first aspect of the invention. The quaternary ammonium compound may be a liquid, although it may be possible to utilise a solid quaternary ammonium compound. The substance may be a liquid or a solid. Suitable reaction conditions, such as the application of heat, may be utilised in order to facilitate the formation of the eutectic liquid. The substance may interact with the anion of the quaternary ammonium compound; for example, the substance may contain at least one hydrogen atom which interacts with the anion of the quaternary ammonium compound through hydrogen bonding.

According to a ninth aspect of the invention there is provided a plasticised polymeric material including an ionic liquid quaternary ammonium compound plasticiser.

Ionic liquids of the invention can be very stable and possess low or zero vapour pressure. It is known that the slow evaporation of prior art plasticisers can lead to polymeric materials becoming brittle and exhibiting cracking as they become older. The use of ionic liquid quaternary ammonium compounds of the invention as plasticisers can address these problems, and is particularly beneficial in polymers that need to have a long working lifetime.

According to a tenth aspect of the invention there is provided the use of an ionic liquid quaternary ammonium compound as a plasticiser in a polymeric material.

According to an eleventh aspect of the invention there is provided a lubricant composition including an ionic liquid quaternary ammonium compound. The lubricant composition may consist of the ionic liquid quaternary ammonium compound, or may include other components. The lubricant composition may be used as a high temperature lubricant, for example at temperatures in excess of 200° C.

According to a twelfth aspect of the invention there is provided the use of an ionic liquid quaternary ammonium compound as a lubricant.

According to a thirteenth aspect of the invention there is provided a method of selectively absorbing a substance from a sample of mixed composition including the steps of contacting the sample with an ionic liquid quaternary ammonium compound so as to selectively absorb the substance.

The sample may be a solid, a liquid, a gas or comprise a mixture of phases, either a simple mixture or a colloidal dispersion.

The sample may be a fuel.

The substance may be a sulphur containing compound and/or a nitrogen containing compound, for example sulphur and/or nitrogen containing impurities in a fuel.

The method may be performed as part of an analytical separation process, which may be a chromatographic technique such as gas chromatography or a liquid chromatography technique.

Whilst the invention has been described above it extends any inventive combination as set out or in the following description and claims.

### EXAMPLE 1

The target molecule 1 is shown below.

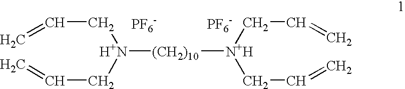

A mixture of 1,10-dibromodecane (23.8 g), diallylamine (15.4 g) and K₂CO₃ (58.0 g) in absolute ethanol were refluxed overnight with a drying arm over the condenser. Reaction progress was checked using TLC. Solid KBr and excess K₂CO₃ were removed from the solvent by filtration. Ethanol was removed by rotary evaporation together with any remaining diallylamine. Any sold KBr appearing at this point in the synthesis can be dissolved in dichloromethane (DCM) and filtered. An ammonium compound was obtained using dry silica gel flushed through with dry DCM. To a solution of the ammonium compound in methanol or dry DCM a 6M aqueous solution of hydroperfluoric acid (HPF₆) is added until the mixture reaches a pH of about 5-6. The water is allowed to evaporate, leaving a quaternary ammonium compound, which was found to be conductive. A conductivity of 100 kΩcm⁻² was recorded.

### EXAMPLE 2

An analogue of the target molecule 1 was prepared in which the anion is replaced by per-fluoro octanoate. The analogue was prepared using the methodology of Example 1, except that aqueous perfluorooctanoic acid was used instead of hydroperfluoric acid. The resulting quaternary ammonium compound exhibited a marginally higher conductivity than the quaternary ammonium of Example 1.

### EXAMPLE 3

An analogue of the target molecule 1 was prepared in which the anion is triflate. The analogue was prepared using the methodology of Example 1, except that triflic acid (CF₃SO₃H) was used instead of hydroperfluoric acid. The analogue exhibited a conductivity of 160 kΩ cm⁻².

The reaction scheme of bromoalkane, diallylamine and K₂CO₃ is a general one that can be used to prepare monomers for subsequent use according to the invention. Bisubstituted bromoalkanes (particularly where the bromo substitution is at either end of the alkyl chain) are used to produce monomers having two dienyl end groups. Singly substituted bromoalkanes are used to produce monomers having one dienyl end group.

1. A method of conduction including the steps of providing a non-polymeric, dienyl quaternary ammonium compound, and causing the quaternary ammonium compound to conduct ionically.

2. A method according to claim 1 in which the quaternary ammonium compound is an ionic liquid.

3.-6. (canceled)

7. A method according to claim 1 in which the quaternary ammonium compound comprises a group of sub-formula (I) 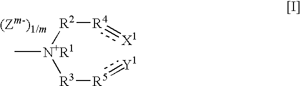
where R² and R³ are independently selected from (CR⁷R⁸)ₙ, or a group CR⁹R¹⁰, CR⁷R⁸CR⁹R¹⁰ or CR⁹R¹⁰CR⁷R⁸ where n is 0, 1 or 2, R⁷ and R⁸ are independently selected from hydrogen, fluoro, halo or hydrocarbyl, and either one of R⁹ or R¹⁰ is hydrogen and the other is an electron withdrawing group, or R⁹ and R¹⁰ together form an electron withdrawing group, and
R⁴ and R⁵ are independently selected from CH or CR¹¹ where R¹¹ is an electron withdrawing group;
the dotted lines indicate the presence or absence of a bond, X¹ is a group CX² X³ where the dotted line bond to which it is attached is absent and a group CX² where the dotted line bond to which it is attached is present, Y¹ is a group CY²Y³ where the dotted line bond to which it is attached is absent and a group CY² where the dotted line bond to which it is attached is present, and X², X³, Y² and Y³ are independently selected from hydrogen and fluorine;
and R¹ is hydrogen or hydrocarbyl, and Z is an anion of charge m.

8.-9. (canceled)

10. A method according to claim 3 wherein the quaternary ammonium compound is a compound of structure (II) 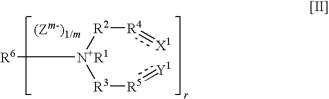
where X¹, Y¹, R², R³, R⁴, R⁵ and the dotted bonds are as defined in claim 7, r is an integer of 1 or more, and R⁶ is a bridging group, an optionally substituted hydrocarbyl group, a perhaloalkyl group, a siloxane group or an amide, of valency r.

11. A method according to claim 4 wherein the quaternary ammonium compound comprises a compound of formula (III) 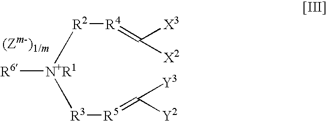
where X², X³, Y², Y³, R², R³, R⁴, and R⁵ are as defined in claim 7, R^{6′} is an optionally substituted hydrocarbyl group, a perhaloalkyl group, a siloxane group or an amide.

12. (canceled)

13. A method according to claim 4 wherein R⁶ or R^{6′} comprises a straight or branched chain alkyl group, optionally substituted or interposed with functional groups.

14. A method according to claim 5 wherein R⁶ or R^{6′} comprises a straight or branched chain alkyl group, optionally substituted or interposed with functional groups.

15.-17. (canceled)

18. A method according to claim 3 in which R¹ is an alkyl group, preferably having less than three carbon atoms, most preferably methyl.

19. A method according to claim 1 in which the quaternary ammonium compound includes a substantially hydrophobic portion.

20.-22. (canceled)

23. A method according to claim 1 in which the quaternary ammonium compound is located in the pores of a porous substrate.

24.-28. (canceled)

29. A method according to claim 1 in which a substance is dissolved in the quaternary ammonium compound.

30.-33. (canceled)

34. A method according to claim 1 in which the quaternary ammonium compound is caused to conduct ionically as part of a fuel cell.

35. A method according to claim 1 in which the quaternary compound is caused to conduct ionically in an electrochemical process.

36. A method according to claim 12 in which the electrochemical process is electroplating, electropolishing or electrowinning.

37. A method of dissolving a substance including the steps of:
providing a liquid, non-polymeric, dienyl quaternary ammonium compound; and
contacting the substance with the said liquid quaternary ammonium compound so that the substance is dissolved in said liquid quaternary ammonium compound.

38.-58. (canceled)

59. A method of coating a surface including the step of contacting a surface with a composition including a liquid, non-polymeric, dienyl quaternary ammonium compound so as to form a coating thereon.

60.-61. (canceled)

62. A structure including a porous substrate and a non-polymeric, dienyl quaternary ammonium compound located in the pores of the porous substrate.

63. A fuel cell including an ionically conductive, non-polymeric, dienyl quaternary ammonium compound.

64.-66. (canceled)

67. A liquid, non-polymeric, dienyl quaternary ammonium compound having a substance dissolved therein.

68.-70. (canceled)

71. A coating composition including a liquid, non-polymeric, dienyl quaternary ammonium compound.

72-73. (canceled)

74. A method of forming an eutectic liquid including the steps of:
providing a non-polymeric, dienyl quaternary ammonium compound; and
mixing said quaternary ammonium compound with a substance so as to produce an eutectic liquid.

75. A plasticised polymeric material including an ionic liquid, non-polymeric, dienyl quaternary ammonium compound plasticiser.

76. (canceled)

77. A lubricant composition including an ionic liquid, non-polymeric, dienyl quaternary ammonium compound.

78. (canceled)

79. A method of selectively absorbing a substance from a sample of mixed composition including the step of contacting the sample with an ionic liquid, non-polymeric, dienyl quaternary ammonium compound so as to selectively absorb the substance.

80.-83. (canceled)
